SEQUENCE LISTING (7 sequences)

SEQ ID NO: 1 - protein, 435 aa, Homo sapiens
MEMEKEFEQIDKSGSWAAIYQDIRHEASDFPCRVAKLPKNKNRNRYRDVSPFDHSRIKLHQEDNDYINASLIKMEEAQRSYILTQGPLPNTCGHFWEMVWEQKSRGVVMLNRVMEKGSLKCAQYWPQKEEKEMIFEDTNLKLTLISEDIKSYYTVRQLELENLTTQETREILHFHYTTWPDFGVPESPASFLNFLFKVRESGSLSPEHGPVVVHCSAGIGRSGTFCLADTCLLLMDKRKDPSSVDIKKVLLEMRKFRMGLIQTADQLRFSYLAVIEGAKFIMGDSSVQDQWKELSHEDLEPPPEHIPPPPRPPKRILEPHNGKCREFFPNHQWVKEETQEDKDCPIKEEKGSPLNAAPYGIESMSQDTEVRSRVVGGSLRGAQAASPAKGEPSLPEKDEDHALSYWKPFLVNMCVATVLTAGAYLCYRFLFNSNT

SEQ ID NO: 2 - protein, 132 aa, Homo sapiens
VDAFLGTWKLVDSKNFDDYMKSLGVGFATRQVASMTKPTTIIEKNGDILTLKTHSTFKNTEISFKLGVEFDETTADDRKVKSIVTLDGGKLVHLQKWDGQETTLVRELIDGKLILTLTHGTAVCTRTYEKEA

SEQ ID NO: 3 - protein, 35 aa, Artificial Sequence
  Exemplary target sequence
AQSYIEKISQAMESAIEKRLTLAQIMEWIRRNIMG

SEQ ID NO: 4 - protein, 35 aa, Artificial Sequence
  Exemplary target sequence
NQSYIELISQAMESAPEKRLTLAQIHEWIRRNAWG

SEQ ID NO: 5 - protein, 35 aa, Artificial Sequence
  Exemplary protein sequence
PYSYISLITMAMQQAPEKMLTLAQIHEWILTHAKP

SEQ ID NO: 6 - protein, 35 aa, Artificial Sequence
  Exemplary protein sequence
PYSYIALITMAMLQSPEKKLTLAQIHEFILVNAKP

SEQ ID NO: 7 - protein, 35 aa, Artificial Sequence
  Exemplary protein sequence
PYSYIELITMAMQNAPEKKITLAQIHQFILVQAKP

(19) United States
(12) Patent Application Publication
(10) Pub. No.: US 2002/0119496 A1
(43) Pub. Date: Aug. 29, 2002

(54) Nuclear magnetic resonance screening method

(76) Inventors: Johan Weigelt, Stockholm (SE); Mats Wikstrom, Stockholm (SE)

Correspondence Address: JANIS K. FRASER, PH.D., Fish & Richardson P.C., 225 Franklin Street, Boston, MA, 02110-2804, US

(21) Appl. No.: 09/986,240

(22) Filed: Oct. 19, 2001

Related U.S. Application Data
(60) Provisional application No. 60/243,626, filed on Oct. 26, 2000.

(30) Foreign Application Priority Data
Oct. 20, 2000 (SE) 0003811-7

(51) Int. Cl. G01N 33/53

(52) U.S. Cl. 435/7.1

(57) ABSTRACT

The invention refers to a method for identifying at least one binder molecule comprising the steps of: (a) choosing two amino acid types (AA1 and AA2) in a polypeptide or protein of interest, whereby AA2 at least once occurs directly subsequent to AA1 in the amino acid sequence of the polypeptide or protein, defining an amino acid pair AA1-AA2; (b) labeling the two amino acid types (AA1 and AA2) in the polypeptide or protein of interest, whereby all AA1-residues is labeled with ¹³C and all AA2-residues with ¹⁵N; (c) generating a first HNCO-type NMR spectrum of the labeled polypeptide or protein from step (b), thereby identifying signals from the labeled amino acid pair AA1-AA2; (d) contacting the labeled polypeptide or protein with a potential binder molecule or a mixture of binder molecules under conditions and sufficient time for allowing binding of the potential binder molecule(s) and the labeled polypeptide or protein; (e) generating a second HNCO-type NMR spectrum, or a ¹H-¹⁵N correlation type NMR spectrum, of the mix from step (d), monitoring signals identified in step (c); (f) comparing the first and the second NMR spectra, whereby a chemical shift change of the signals identified in step (c) between the two spectra indicates an interaction between the potential binder molecule and the labeled polypeptide or protein.

### CROSS REFERENCE TO RELATED APPLICATIONS

This application claims priority from Swedish Patent Application No. 0003811-7, filed Oct. 20, 2000, and U.S. Provisional Patent Application Serial No. 60/243,626, filed Oct. 26, 2000. These applications are incorporated herein by reference in their entirety.

### TECHNICAL FIELD

The present invention relates to a nuclear magnetic resonance (NMR) based method for assaying binding of various chemical compounds to a polypeptide or a protein. More specifically, the method allows for the screening of binding to a designated binding epitope on the surface of the polypeptide or protein.

### TECHNICAL BACKGROUND

In modern biology and medicine, there has been a demand for physical methods, which can make it possible to study the structure of small and large biomolecules, as well as the interaction between various molecules and compounds. For this purpose, several powerful techniques have been applied, such as x-ray crystallography, mass spectrometry, and nuclear magnetic resonance (NMR).

In recent years, NMR spectroscopy has become an important tool in the drug discovery process through the advent of NMR based screening methods to identify lead templates. Several techniques have been introduced, [Shuker, 1996; Chen, 1998; Mayer, 1999; Chen, 2000; Jahnke, 2000] perhaps the most well known is the “SAR by NMR” method described by Fesik and coworkers in 1996. [WO97/18471, Shuker, 1996 and EP-B1-0866967] The SAR by NMR technique relies on detecting chemical shift changes in a two-dimensional ¹H-¹⁵N correlation spectrum to identify compounds that bind to the target protein. When a first ligand has been identified, a second ligand is sought for in the presence of saturating concentrations of the first ligand (screen for “second-site” binder). Provided the three dimensional structure of the protein is known and sequence specific NMR assignments of the protein backbone resonances have been obtained, the two ligands may be linked based on structural data. To first approximation the binding affinity of the linked compound will be the product of the binding affinities of the individual compounds, resulting in high affinity “nanomolar binders” even if the starting compounds only had millimolar to micromolar affinities. [Shuker, 1996]

A prerequisite for the SAR by NMR method is that sequence specific resonance assignments have been obtained for the backbone NMR resonances (¹⁵N, ¹³C_{α}, ¹³C′, ¹H^{N}) of the target protein. This is a formidable task that demands several months of experimental work and data analysis even for a relatively small protein.

For structural studies of large proteins, Yabuki et al., 1998, discloses a method, which method involves the site-specific labeling of two in the peptide sequence following amino acids with ¹³C and ¹⁵N, respectively. This is done in a so-called cell-free system, in order to avoid problems of disturbing incorporations. Hereby, due to 1J_{CN}-coupling it is possible to study only one signal in an adequate spectrum. This article is mainly focused on the development of methods for cell-free synthesis for structural studies of the human c-Ha-Ras protein, as well as protein-protein interactions.

Accordingly, the known techniques for screening for small binder molecules to a specific site in a target protein, such as an active site, pose some disadvantages, in that they are time-consuming and involve a lot of experimental work as well as complex interpretation of data achieved. Thus, there is a need for a method allowing identification of binder molecules to a specific target in an easier and more effective way, thereby limiting the experimental work.

The object of the invention is to reduce the drawbacks and expand the possibilities of the prior art.

### SUMMARY OF THE INVENTION

This object is, according to the invention, solved by a method for identifying at least one binder molecule comprising the steps of:

(a) choosing two amino acid types (AA1 and AA2) in a polypeptide or protein of interest, whereby AA2 at least once occurs directly subsequent to AA1 in the amino acid sequence of the polypeptide or protein, defining an amino acid pair AA1-AA2;

(b) labeling the two amino acid types (AA1 and AA2) in the polypeptide or protein of interest, whereby all AA1-residues is labeled with ¹³C and all AA2-residues with ¹⁵N;

(c) generating a first HNCO-type NMR spectrum of the labeled polypeptide or protein from step (b), thereby identifying signals from the labeled amino acid pair AA1-AA2;

(d) contacting the labeled polypeptide or protein with a potential binder molecule or a mixture of binder molecules under conditions and sufficient time for allowing binding of the potential binder molecule(s) and the labeled polypeptide or protein;

(e) generating a second HNCO-type NMR spectrum, or a ¹H-¹⁵N correlation type NMR spectrum, of the mix from step (d), monitoring signals identified in step (c);

(f) comparing the first and the second NMR spectra, whereby a chemical shift change of the signals identified in step (c) between the two spectra indicates an interaction between the potential binder molecule and the labeled polypeptide or protein.

Hereby, by using efforts from the structural studies of large proteins in the field of drug molecule screening, the inventors have unexpectedly provided a method, which allows an easy identification of binder molecules to a target molecule.

Yet another aspect of the invention is a method, whereby the labeled amino acid pair AA1-AA2 is unique within a sphere radius of 10 Å, preferably 50 Å, and most preferably within the whole polypeptide or protein.

One embodiment of the invention is a method, whereby the labeled amino acid pair AA1-AA2 is within a binding pocket of the polypeptide or protein. This allows for the screening for binding to a single binding site. The requirement is that a unique amino acid pair occurs in the binding pocket.

Another embodiment of the invention is a method, whereby the labeled amino acid pair AA1-AA2 is in the proximity, preferably closer than 15 Å, of an active site within the polypeptide or protein. This enables the screening for specificity in vicinity of an active site in the polypeptide or protein. If the target protein has one or more unique amino acid pairs in the vicinity of an active site, whereby the unique amino acid pairs differs from other members of the same protein family, this can be used to screen for selectivity, i.e. the different proteins of the same family can be compared. Different compounds may give different chemical shift, why the longest distance from the active site, which makes specificity screening possible, varies.

Yet another embodiment of the invention is a method, whereby the result of the method is compared to the result of any other suitable binding or activity assay, such as a fluorescence-based assay, a reporter gene assay, displacement assays or ELISA. This allows for a rapid confirmation of the binding to this specifically labeled site, or as a selection of candidates for more extensive study by any other suitable method.

Accordingly, according to another aspect of the invention, the method is used for screening of a compound library.

All publications, patent applications, patents, and other references mentioned herein are incorporated by reference in their entirety.

### SHORT DESCRIPTION OF THE DRAWINGS

FIG. 1 shows the site specific labeling of the PTP1B-protein. All arginine and aspartate residues have been enriched with ¹³C and ¹⁵N, respectively. There is only one unique Arg-Asp pair in the sequence (boxed).

FIG. 2 shows 2D NMR spectra of ¹⁵N-Asp, ¹³C-Arg labeled PTP1B: (A) 2D TROSY of free protein; (B) 2D HNCO of free protein; and (C-F) 2D TROSY spectra of (C) free protein; (D) protein+compound cocktail; (E) protein+compound cocktail omitting PNU179983; and (F) protein+PNU179983.

FIG. 3 is a ribbon drawing of PTP1B showing the five selectively labelled amino acid residue pairs. The “Y-loop pairs” Arg47-Asp48 (I) and Arg43-Asn44 (II) are shown, as well as Arg156-Gln157 (III), Arg 169-Glu170 (IV) and Arg199-Glu200 (V), and the active site cysteine (VI).

FIG. 4 is a table showing the result of a colorimetric assay. All compounds were tested in a clorimetric assay. Samples containing 1 μM PTP1B, 2 mM paranitrophenylphosphate (substrate) and 1 mM compound were prepared in 20 mM Tris-HCl buffer at pH 7.5 and incubated at room temperature for 5 minutes. The reaction was stopped by addition of NaOH to raise the pH. The amount of inhibition was calculated from the measured absorbance of the resulting product (para-nitrophenole).

FIG. 5 is a stereo-view of PNU179983 binding to PTP1B as determined from a preliminary x-ray crystallographic electron density map obtained at 2.2 Å resolution.

PNU179983 clearly interact not only with the active residues but also have close contacts with the Y-loop (highlighted) via hydrogen bonds. The closest distance between PNU179983 ant the Y-loop is approximately 2.8 Å. The crystals were grown in the presence of 2 mM inhibitor in 0.1 M pH 6.5 cacodylate buffer, 0.2 M Mg acetate, 16-20% PEG 8000. The crystals belonged to space group P222 with cell dimensions a=53.2 Å, b=84.3 Å, c=88.7 Å, alpha=beta=gamma=90. Data extended to 2.2 Å with Rsym=6.7%. Data was processed and the model refined to R=25%.

FIG. 6 is a schematic view of one embodiment of the invention (screening for binding to a single binding site). An exemplary target sequence is shown (SEQ ID NO:3).

FIG. 7 is a schematic view of another embodiment of the invention (screening for specificity in vicinity of an active site). An exemplary target sequence, and exemplary protein sequences x, y, and z are also shown (SEQ ID NOs:4-7, respectively).

FIG. 8 shows a NMR-spectra recorded on a 200 μM 13C′-Val, 15N-Ala selectively labeled sample of M-FABP. The spectra were recorded at 20° C. on a 600 MHz Varian Unity INOVA NMR spectrometer using standard techniques. The sample was prepared in 20 mM sodium phosphate buffer (10% D₂O/90% H₂O) pH 7.4 containing 50 mM NaCl, 10 mM DTT and 0.02% NaN₃. NmrPipe (Delaglio et al., 1995) was used for data processing. (a) 2D ¹H-¹⁵N fast-HSQC (Mori et al., 1995), (b) 2D HNCO and (c) 1D HNCO. The HNCO spectra in panels b and c identify the resonance corresponding to alanine 33 (Roman numeral II in panel a). Experimental times were 1 hour, 4 hours, and 20 minutes respectively.

FIG. 9 shows plots of ¹H-¹⁵N fHSQC spectra for M-FABP recorded at 600 MHz showing the region corresponding to the alanine 33 cross peak of FIG. 8a. Experimental conditions were identical to those described in FIG. 8 except the samples used for spectra b-d contained additionally 1% d-DMSO. (a-d) Reference spectrum of free M-FABP. (b) 1:1 mixture of M-FABP and compound cocktail. (c) 1:1 mixture M-FABP and compound cocktail omitting oleic acid. (d) 1:1 mixture of M-FABP and oleic acid.

### DETAILED DESCRIPTION OF THE INVENTION

By a “compound library” is meant a set of chemical compounds, that for example can be used for assaying binding to a macromolecule. The compound library may comprise from about 2-100.000 compounds, preferably 100-1000. The compounds of the library can be of any size, preferably 50-1000 D. The compounds can be any organic molecules or natural products.

By a “polypeptide or protein of interest” is meant any amino acid sequence, or a complex of amino acid sequences, having a potential binding epitope.

By a “potential binder molecule” is meant a chemical compound, which may bind to the polypeptide or protein of interest, and which may be a member of the compound library. The potential binder molecule may be a peptide, a polypeptide, a protein, an antibody, a nucleic acid molecule, a carbohydrate, or a part or a complex of one or more of the mentioned molecule types, or any other chemical compound of interest. Preferably, the potential binder molecule is a relatively small molecule, such as a molecule in the size interval of 50-1000 D.

“AA1” and “AA2” can be any one of the about 20 different naturally occurring amino acid types. Furthermore, AA1 and/or AA2 may also be any modified variant of an amino acid, as long as it is possible to detect the 1J_{CN}-coupling. AA1 and AA2 may be the same or different type.

By AA2 at least once occurs “directly subsequent” to AA1 in the amino acid sequence is meant that AA1 in this case has the sequence position n−1 and AA2 sequence position n.

A “AA1 -AA2-pair” is a pair of amino acids following each other directly in sequence, whereby AA1 has the number n−1 and AA2 the number n in the amino acid sequence.

By a “binding pocket” is meant an epitope on the polypeptide or protein of interest, that is expected to allow binding of a potential binder molecule.

By a “sphere radius” in this context is meant a defined distance in any direction in space from a defined pair of amino acids (AA1-AA2).

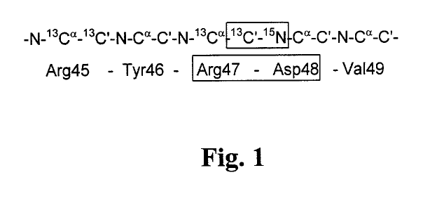

The core of the invention is a revitalization of a sequence specific labeling method that has not been extensively used. [Kainosho, 1982, Kato, 1991, Yabuki, 1991] All amino acids AA1 are labeled with ¹³C and all amino acids AA2 are labeled with ¹⁵N. Provided only one AA1-AA2 pair occurs in the amino acid sequence, only one signal in the 1D carbonyl ¹³C spectrum will display a splitting due to the ¹J_{CN} coupling. Obviously only one peak will appear in a 1D (or 2D or 3D) HNCO type correlation spectrum (FIG. 1).

The labeling strategy is only sequence specific in an indirect sense, i.e. the occurrence of a unique pair of labeled amino acid residues confers the sequence specificity. Using this technique it is possible to screen selectively for binding to a selected epitope without the need for sequence specific assignments. The HNCO spectrum can thus be used either directly as a screening experiment (1D or 2D versions) or indirectly to identify what signals to monitor in a 2D ¹H-¹⁵N correlation spectrum. Chemical shift perturbations upon addition of a potential ligand are easily detected even for large proteins due to the reduced spectral complexity resulting from the use of a selectively labeled sample.

The site specificity is inherent to the labeling technique and the key is to find a unique sequence motif in the binding site that is to be screened. Given a protein of 300 amino acid residues, and assuming equal and random distribution of all 20 amino acid residues, the probability that a given amino acid residue pair only occurs once in the sequence is approximately 0.5. If the desired site contains at least 3-4 suitable pairs, the probability of finding a unique pair is reasonably high, i.e. 85-95%.

The most reliable way to obtain a sequence specific labeled protein is to over-express the protein using cell-free synthesis as described by Yabuki et al. [Yabuki, 1998]. Alternatively the protein could be over-expressed in rich medium containing the labeled amino acids, using a bacterial strain with gene lesions suitable for the particular type of selective amino acid enrichment [Muchmore, 1989]. In favorable cases however, good results have been reported using a prototrophic bacterial strain in a rich medium containing all amino acids and nucleotides. [Kainosho, 1982] [Muchmore, 1989]

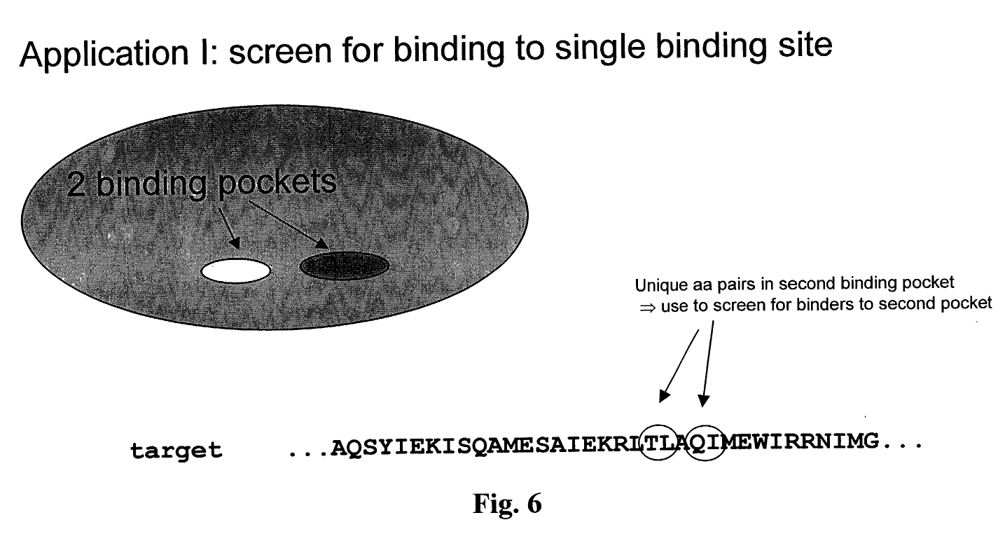

According to one embodiment of the invention, the method can be used for screening for binding to a single binding site (FIG. 6). This can be done if the target protein or polypeptide of interest has at least one unique amino acid pair (two amino acids being adjacent to each other in the primary structure) within the potential binding site. Then, this unique amino acid pair is labeled according to the method of the invention, and the interaction between the binding site and potential binder molecules can be studied.

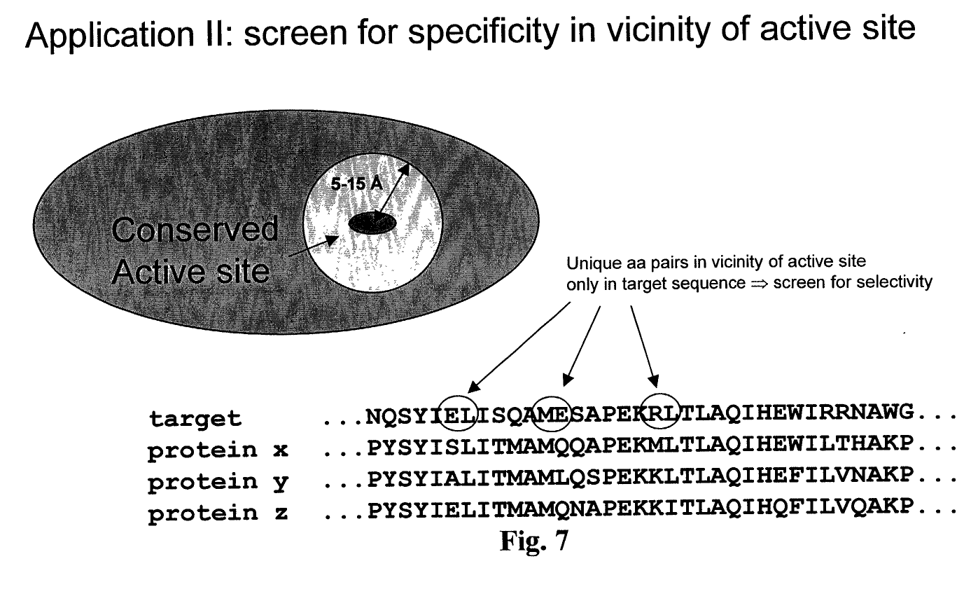

According to another embodiment of the invention, the method can be used for screening for specificity in vicinity of an active site (FIG. 7). This can be achieved if the target protein or polypeptide of interest has at least one unique amino acid pair in vicinity of the active site (preferably closer than 15 Å, and more preferably within 5-15 Å). Further, if the target protein differs from other proteins within the same protein family in that the target protein has at least one unique amino acid compared to the other proteins, this can be used to screen for selectivity of potential binder molecules to the target protein. This means that the unique amino acid, which differs from the other proteins of the family, may be close to the active site with regard to tertiary structure, but not necessarily close with regard to primary structure. However, the unique amino acid should preferably not be within the active site.

According to yet another embodiment of the invention, the method of the invention can be used in combination with any other suitable binding or activity assay, such as a fluorescence based assay, a reporter gene assay, displacement assays or ELISA, in order to either confirm the result of that method, or to screen for interesting compounds, which subsequently are more thoroughly studied by another method. The method of the invention can be advantageously used in this aspect as it provides a method, which rapidly gives a reliable result. This can be useful when there are no X-ray data for the studied protein, but only computer modelling data.

The conditions suitable for allowing the potential binder molecule and the labeled protein or polypeptide of interest to interact, as well as to monitor the interaction by NMR, are standard conditions for protein NMR [Cavanagh] (buffered solutions, pH kept stable, reaction temperature 5-50° C.). Preferably, the target protein concentration may range from 25 μM-1 mM, and the potential binder molecule concentration from 25 μM-1 mM.

In order for a change in the chemical shift to be considered relevant, the change should be equal to or greater than the natural line width, or the signals should be exchange-broadened beyond detection.

Moreover, the method of the invention may be used for competition binding analysis experiments.

To summarize, the new technique presents a new approach to screen and identify binders to protein targets in a site-specific manner. This will allow the identification of scaffolds (by screening a small compound library) with a binding preference for a particular site or sites, which could confer specificity for a certain target within a protein family, and also confirm the binding mode from hits detected using other suitable binding or activity assays, such as a fluorescence based assay, a reporter gene assay, displacement assays or ELISA.

Also, the new technique has the potential to allow NMR studies of proteins of much larger size than traditional SAR-BY-NMR since essentially no assignment of the protein resonances has to be undertaken. Conventionally, only proteins of the size up to approximately 30 kD are possible to study. With the method of the invention, proteins of the size 50 kD, and most probably of the size up to 100 kD, can be monitored. The sample preparation can be prepared according to previously described protocols. [Muchmore 1989, Yabuki 1998]

For further details on NMR spectroscopy or on experimental approaches relevant in the field of the invention, WO97/18471 and Cavanagh, 1996 are hereby incorporated as references.

Below, the invention is described by the following examples, which only are to be seen as exemplifying the invention, and not limiting the scope of the invention in any way.

### EXAMPLES

### Example 1

Screening for Binding to the Y-Loop of Protein Tyrosine Phosphatase-1B (PTP1B) (SEQ ID NO:1)

PTP1B (35 kD, single domain protein] is a protein that dephosphorylates phosphotyrosines. The active site binding cleft is centered around an active cysteine residue. It has been speculated that the so-called Y-loop of PTP1B is important for binding of some ligands. The Y-loop contains a sequence motif that is unique in the sequence, i.e. Arg47-Asp48 (FIG. 1). A selective labeled sample of PTP1B was prepared to monitor binding to this site.

Site specific labeled PTP1B (residues 1-298) was prepared from transformed Escherichia coli strain BL21 (DE3) cells. Bacteria were grown in rich medium containing all amino acids and 5 nucleotides according to the protocol described by Muchmore et al. [Muchmore, 1989] Aspartate and arginine were supplied ¹⁵N-enriched and ¹³C-enriched, respectively. It should be noted that this protocol (i.e. the use of a prototrophic bacterial strain) for preparing the sample is not optimal for the production of selectively ¹⁵N-Asp enriched protein. Aminotransferase activity may cause misincorporation of ¹⁵N in other amino acid residues. For the case of Asp, incorporation in Asn, Glu and Gln residues may be seen. [Muchmore, 1989] Nevertheless, the risk of misincorporation of the ¹⁵N label is in no way a limitation to the invention. A selectively labeled protein may be produced with no misincorporation of the ¹⁵N (or ¹³C) label if a bacterial strain with gene lesions suitable to the particular type of selective amino acid enrichment is used. [Muchmore, 1989] Alternatively, the sample could be prepared using cell-free synthesis [Yabuki, 1998]. It should also be noted that in favorable cases good results have been reported also when prototrophic bacterial strains were used. [Kainosho, 1982][Muchmore, 1989]

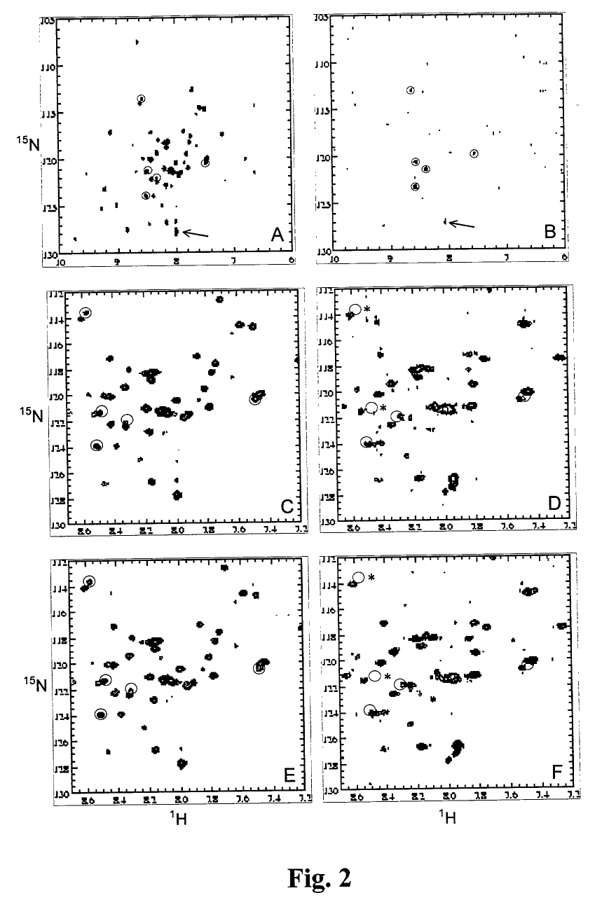

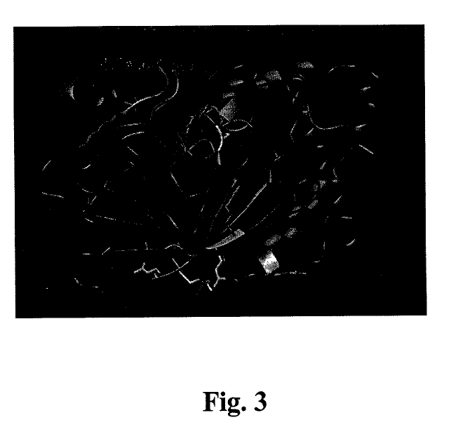

A two-dimensional ¹H-¹³C correlation spectrum of the selectively labeled sample verified that only Arg residues were ¹³C-enriched. The corresponding ¹H-¹⁵N experiment indicated, however, that ¹⁵N was not exclusively incorporated in Asp residues. FIG. 2A shows the 2D ¹H-¹⁵N TROSY [Weigelt, 1998] spectrum of the ¹⁵N-Asp, ¹³C-Arg sample. If only Asp residues were ¹⁵N-labeled a total of 18 peaks would be expected in this ¹H-¹⁵N correlation spectrum (18 Asp residues in the amino acid sequence). Instead the spectrum contains approximately 65 peaks, which is consistent with incorporation of ¹⁵N also at Asn, Glu and Gln sites (totally 69 in the amino acid sequence). Studying the amino acid sequence in detail one can see that there now instead of one unique pair, Arg47-Arg48, are four additional ones, Arg43-Asn44, Arg156-Gln157, Arg169-Glu170, and Arg199-Glu200. The first of these, Arg43-Asn44, is located in the beginning of the Y-loop. The other three are situated on the protein surface far from the binding site (FIG. 3).

FIG. 2B shows the 2D HNCO [Cavanagh, 1996] spectrum that selects signals from the selectively labeled pairs. As expected, five peaks corresponding to the five unique sequence pairs appear in the spectrum (circled). An additional peak (marked with an arrow) originating from the C-terminal Asp residue is also seen. The natural abundance ¹³C (1%) of the preceding Glu residue is enough to yield an HNCO signal due to the extremely strong H^{N}-resonance of the C-terminal Asp residue.

To detect binding to the Y-loop one would expect that one or two of the designated signals would experience large chemical shift changes upon addition of a compound that interacts with the Y-loop, whereas the other three signals would remain essentially unaffected.

To test this hypothesis the inventors recorded 2D TROSY spectra in the presence of a cocktail of five compounds. One of the compounds, N200, was known to bind to the protein as determined by a NMR-line broadening assay (more than 5 Hz line broadening at 50 μM concentration in the presence of an equimolar amount of PTP1B). Three compounds, N35, N136 and N212, were non-binders as determined by NMR. The fifth compound, PNU179983, was a micromolar inhibitor of PTP1B believed to interact with the Y-loop. FIG. 2C shows a selected area from the TROSY spectrum of free ¹⁵N-Asp, ¹³C-Arg PTP1B. FIG. 2D shows the spectrum in presence of the cocktail. FIG. 2E shows the spectrum in presence of all compounds from the cocktail except PNU179983. FIG. 2F shows the spectrum with only PNU179983 present. In all spectra the signals corresponding to the HNCO selected signals are circled.

It is evident that substantial spectral changes occur in the presence of the compound cocktail (FIG. 2D). In particular two of the designated peaks (marked with asterisks) disappear altogether, whereas the other three signals only experience minor shifts. This indicates that at least one of the compounds in the cocktail interacts strongly with the Y-loop. When PNU179983 was omitted from the cocktail (FIG. 2E) no shifts are observed for any of the designated signals. FIG. 2F shows that PNU179983 is responsible for the large shifts since this spectrum shows the same pattern as the one recorded in presence of the full cocktail (FIG. 2D).

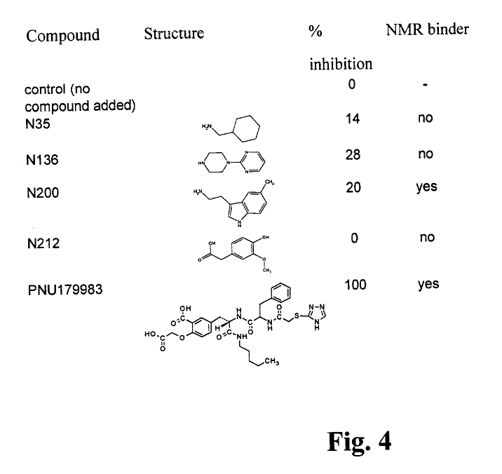

To further substantiate the finding that the invention allows identification of binders to a selected site all compounds were tested in a colorimetric assay (FIG. 4). N 200, which binds to PTP1B according to the NMR line-broadening assay, showed partial inhibition of the enzyme, indicating binding to the active site. N35 and N136 also showed partial inhibition, which would indicate that they were false negatives or interacting too weakly to indicate binding in the NMR line-broadening assay. PNU179983 showed complete inhibition of the enzyme.

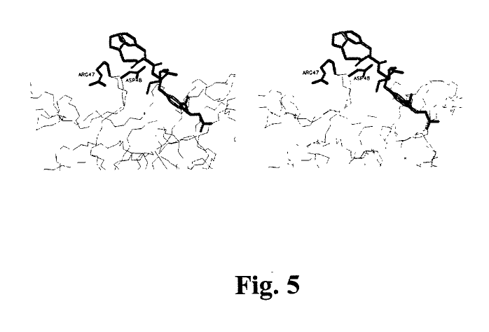

Furthermore, an x-ray crystallographic study was performed to assess the binding mode of PNU179983. Preliminary x-ray crystallographic data at 2.2 Å resolution (Derek Ogg, unpublished results) showed that PNU179983 binds to PTP1B with interactions not only within the active site but also with the Y-loop (FIG. 5). The inhibitor is in close proximity to the Y-loop and interacts with the loop via hydrogen bonds. The shortest distance between the inhibitor and the Y-loop is approximately 2.8 Å.

### Example 2

Site Selective Screening of Human Muscle Fatty Acid Binding Protein (M-FABP)(SEQ ID NO:2).

The principle of the site-selective screening method is demonstrated for the human muscle fatty acid binding protein M-FABP. A 1.4 Å X-ray crystal structure of M-FABP in complex with a ligand, oleic acid, is available (PDB ID code: 1 HMS). [Young, 1994] The structure consists of 10 anti-parallel β-strands and two α-helices that connect β-strands 1 and 2. The β-strands form two nearly orthogonal β-sheets. The fatty acid binding site is situated in a cavity between the two β-sheets. The cavity is also lined by residues from helices 1 and 2. The carboxylate group of the fatty acid forms hydrogen bonds (direct and solvent mediated) to the protein. The aliphatic tail of the fatty acid adopts a u-shaped conformation in the highly hydrophobic cavity. Alanine 33 makes contacts with carbons C12, C13 and C14 of the oleic acid. [Young, 1994] Valine 32 and alanine 33 comprise a unique amino acid residue pair in the amino acid sequence, and are thus suitable for selective labeling. It is likely that the NMR resonances of alanine 33 will be affected upon binding of oleic acid. A 13C′-Val, 15N-Ala selectively labeled sample of M-FABP was produced from prototrophic Escherichia coli cells harboring a plasmid containing the gene coding for a 143 amino acid protein construct of M-FABP. The construct encompassed a C-terminal his-tag included for purification purposes. Cells were grown in a fermentor using a growth medium containing all amino acids and nucleotides according to the protocol of Muchmore et al. Valine was supplied with the carboxyl carbon 13C-labeled, alanine was supplied 15N labeled (Cambridge Isotope Laboratories). Muchmore et al. advocates the use of auxotrophic bacterial strains with gene deletions suitable for the particular type of amino acid labeling that is desired. Even better performance is expected from cell-free synthesis [Yabuki, 1998]. Good results have, however, been reported also when prototrophic bacterial strains were used. [Kainosho, 1982; Kato, 1991; Muchmore, 1989; Senn, 1987] The protein was purified by affinity chromatography using a Ni2+ charged 5 ml HiTrap Chelating column (Amersham Pharmacia Biotech), followed by a Lipidex-1000 column (Sigma) as described by Constantine et al.

The protein mass was determined using Electrospray Mass Spectrometry. The extent of incorporation of ¹³C and ¹⁵N in valine and alanine residues was estimated to be at least 95%.

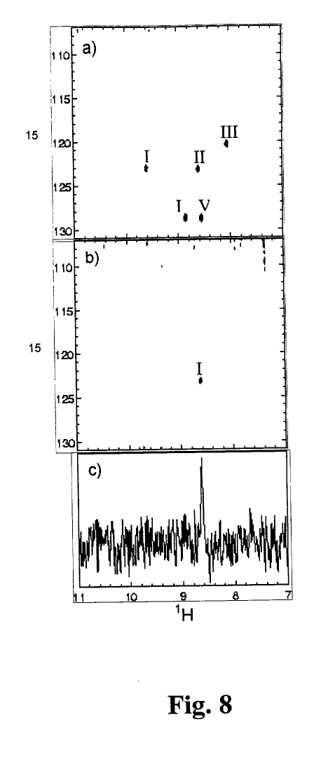

FIG. 8 shows NMR spectra of selectively labeled M-FABP. The signals visible in a ¹H-¹⁵N correlation experiment (FIG. 8a) correspond perfectly to the alanine resonances of M-FABP. [Constantine, 1998] The 2D HNCO experiment yields cross peaks only for ¹³C-¹⁵N-¹H moieties. There is only one such instance in the selectively labeled M-FABP protein, namely (¹³C′)Val32-(¹⁵N)Ala33. The HNCO spectrum in FIG. 8b contains only one cross peak which readily identifies the resonance corresponding to alanine 33, in agreement with the published resonance assignment. The spectrum displayed in FIG. 8c demonstrates the feasibility of the use of a 1D HNCO experiment for detection.

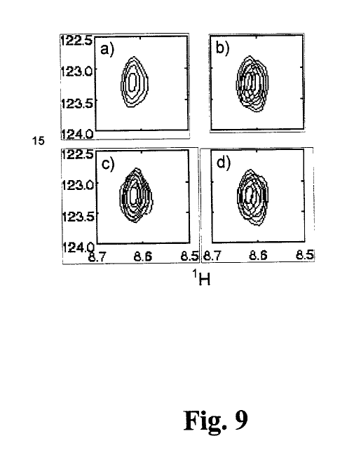

Five test compounds—oleic acid and four compounds from our NMR screening library showing no NMR line broadening when mixed with M-FABP—were mixed into a compound cocktail. FIG. 9 displays spectral expansions of spectra recorded on selectively labeled M-FABP in different mixtures.

As expected the HSQC cross peak belonging to alanine 33 experiences a chemical shift change upon addition of the test cocktail (FIG. 9b). The same chemical shift change is observed when M-FABP is mixed with oleic acid alone (FIG. 9d). Omitting oleic acid from the test cocktail leaves the spectrum unchanged (FIG. 9c). Clearly the site-selective labeling method promises to be a valuable tool for identifying compounds with specific binding properties. Potential applications include: screening for binders to a selected site that has been identified either through x-ray, NMR or modeling studies; an assay to confirm binding of ligands identified by other methods (i.e. HTS) to a desired site; screening for ligands that bind to a site that could confer binding specificity for one target protein within a protein family. Due to the reduced spectral complexity resulting from the use of a selectively labeled sample, the method should be applicable to larger proteins than are conventional methods.

### REFERENCES

Cavanagh et al., “Protein NMR spectroscopy: principles and practice” (1996), Academic Press, San Diego.

Chen and Shapiro J. Am. Chem. Soc. (1998), 120, 10258-10259.

Chen and Shapiro J. Am. Chem. Soc (2000), 122, 414-415.

Constantine et al., Biochemistry 1998, 37, 7965-7980.

Delaglio et al., J. Biomol. NMR 1995, 6, 277-293.

Jahnke et al. J. Am. Chem. Soc. (2000), 122, 7394-7395.

Kainosho and Tsuji Biochemistry (1982), 21, 6273-6279.

Kato et al. Biochemistry (1991), 30, 270-278.

Mori et al., J. Magn. Reson. 1995, B108, 94-98.

Muchmore et al Methods in Enzymology (1989), 44-73.

Mayer and Meyer Angew. Chem. Int. Ed (1999), 38, 1784-1788.

Senn et al., Eur. Biophys. J., 1987, 14, 301-306.

Shuker et al. Science (1996), 274, 1531-1534.

Weigelt, J. Am. Chem. Soc. (1998), 120, 10778-10779.

Yabuki et al., Journal of Biomol. NMR, 1998, 11, 295-306.

Young et al., Structure 1994, 2, 523-34.

What is claimed is:

1. A method for identifying at least one binder molecule comprising the steps of:
(a) choosing two amino acid types (AA1 and AA2) in a polypeptide or protein of interest, whereby AA2 at least once occurs directly subsequent to AA1 in the amino acid sequence of the polypeptide or protein, defining an amino acid pair AA1-AA2;
(b) labeling the two amino acid types (AA1 and AA2) in the polypeptide or protein of interest, whereby all AA1-residues is labeled with ¹³C and all AA2-residues with ¹⁵N;
(c) generating a first HNCO-type NMR spectrum of the labeled polypeptide or protein from step (b), thereby identifying signals from the labeled amino acid pair AA1-AA2;
(d) contacting the labeled polypeptide or protein with a potential binder molecule or a mixture of binder molecules under conditions and sufficient time for allowing binding of the potential binder molecule or the binder molecules and the labeled polypeptide or protein;
(e) generating a second HNCO-type NMR spectrum, or a ¹H-¹⁵N correlation type NMR spectrum, of the mix from step (d), monitoring signals identified in step (c); and
(f) comparing the first and the second NMR spectra, whereby a chemical shift change of the signals identified in step (c) between the two spectra indicates an interaction between the potential binder molecule and the labeled polypeptide or protein.

2. The method of claim 1, wherein the labeled amino acid pair AA1-AA2 is unique within a sphere radius of 10 Å within the polypeptide or protein.

3. The method of claim 1, wherein the labeled amino acid pair AA1-AA2 is unique within a sphere radius of 50 Å within the polypeptide or protein.

4. The method of claim 1, wherein the labeled amino acid pair AA1-AA2 is unique within the polypeptide or protein.

5. The method of claim 1, wherein the labeled amino acid pair AA1-AA2 is within a binding pocket of the polypeptide or protein.

6. The method of claim 1, wherein the labeled amino acid pair AA1-AA2 is in the proximity of an active site within the polypeptide or protein.

7. The method of claim 1, wherein the result of the method is compared to the result of any other suitable binding or activity assay.

8. The method of claim 1, wherein the polypeptide or protein has a size of from 10 to 150 kDa.

9. The method of claim 1, wherein the potential binder molecule is a peptide, polypeptide, protein, antibody, nucleic acid molecule, or carbohydrate.

10. The method of claim 1, wherein the potential binder molecule comprises a complex of one or more of a peptide, polypeptide, protein, antibody, nucleic acid molecule, or carbohydrate.

11. The method of claim 1, wherein the potential binder molecule has a molecular mass of from 50 to 1000 Da.

12. The method of claim 1, wherein the method is used for screening a compound library.
